(19) United States
(12) Patent Application Publication
(10) Pub. No.: US 2002/0018989 A1
(43) Pub. Date: Feb. 14, 2002

(54) METHOD OF IDENTIFYING PHARMACEUTICALS FROM PLANT EXTRACTS

(76) Inventors: ALBERT BEAUFOUR, LONDON (GB); JACQUES-PIERRE MOREAU, UPTON, MA (US)

Correspondence Address: BRIAN R. MORRILL, ESQ., BIOMEASURE INCORPORATED, 27 MAPLE STREET, MILFORD, MA, 01757-3650, US

(21) Appl. No.: 08/744,983

(22) Filed: Nov. 7, 1996

(51) Int. Cl. C12Q 1/68; C07H 21/04; C07H 21/02; C12P 19/34

(52) U.S. Cl. 435/6; 435/91.2; 536/23.1

(57) ABSTRACT

The invention features a method of identifying a pharmaceutical comprising compounds found in a plant extract by utilizing a genomic screen of the plant extract.

### BACKAROUND OF THE INVENTION

The microbial, plant, and animal (e.g. invertebrates) kingdoms constitute rich natural repositories of active ingredients with varied physico-chemical and medicinal properties. The U.S. and European pharmacopeias are replete with examples of medicaments derived from natural sources. See U.S. Pharmacopeia 1995 (United States Pharmacopeial Convention, Inc., 1994) and Martindale, The Extra Pharmacopeia 31 (Royal Pharmaceutical Society, 1996). Many antimicrobial agents (e.g., penicillins, cephalosporins, and aminoglycosides), antifungals (e.g., amphotericin B and nystatin), anti-parasitics (e.g., quinine), cardio- and vaso-active agents (e.g., cardiac glycosides-digoxin, ergot alkaloids, nicotine, and oxytocin), anti-inflammatory agents (e.g., aspirin), muscarinic (e.g., acetylcholine) and antimuscarinic (e.g., atropine and scopolamine) agents, neuroactive agents (e.g., curare, physostigmine, and opiates), anticoagulants (e.g., heparin), antineoplastic agents (e.g., vinca alkaloids, taxol, and podophyllotoxin derivative etoposide), and hormones (e.g., estrogens, androgens, progestins, peptide hormones, and growth factors) were discovered as natural products. See, e.g., Goodman & Gilman's The Pharmacological Basis of Therapeutics (9th Edition McGraw-Hill, 1996). These examples suggest that some active components in plant extracts may also be purified and still retain their biological potency.

In most of the above examples, the salient ethnopharmacological properties of the compound were characterized in known pharmacological systems and assays only upon isolation of the compound from the natural source. See, e.g., Turner, R. A. Screening Methods in Pharmacology (Academic Press, 1965) and Turner, R. A., Peter Hebborn Screening Methods in Pharmacology, (Vol. II, Academic Press, 1971). While the process of determining or substantiating activities of plant extracts may be discovered by the screening of their individual components, many of these extracts may have additional pharmacological activities that may not be evident from the extract's known, if any, ethnopharmacological background of its individual components, (e.g., the extract's ability to affect signal transduction to the nucleus, alter the trafficking of cell surface receptors, and regulate genomic events).

The characterization of defined active natural compounds, either derived from natural sources or produced synthetically or semisynthetically, constitutes the pharmacological basis for much of Western Medicine. The process of distilling an ethnopharmacologically active plant extract down to a single active principal, however, may result in a loss of biological activity for a number of reasons (e.g., a particular compound is unstable during extraction or in the purified form, the compound may react with chemical entities in the extract during purification, the compound is fractioned out during purification, or, more importantly, the fundamental basis for ethnopharmacology does not always reside in a single active compound being present in the extract but rather is a result of the interaction of two or more active compounds found in the extract). Thus, the likelihood that more than one compound present in a plant extract could contribute to a net pharmacological response of the extract, as well as a genomic means to identify such compounds in a natural product, is a novel pharmacological concept.

The present invention provides for a genomic screen in which an extract from a plant may be characterized for its potential biological properties which may or may not be related to the known, if any, ethnopharmacological properties of the extract. The present invention represents a novel approach to the analysis of the potential mechanism(s) of action of plant extracts, as the underlying basis for therapeutic efficacy, and a means for identifying the individual compound(s) which elicit the discovered biological property in order to identify a new pharmaceutical or new pharmaceutical use for an existing pharmaceutical.

### SUMMARY OF THE INVENTION

In one aspect, the invention features a method of identifying a pharmaceutical, the method comprising the steps of: administering a plant extract to a cell type; isolating protein or RNA (e.g., messenger RNA) from the plant extract treated cell type; identifying which of the protein or RNA isolated from the plant extract treated cell type is not present in the same concentration in the untreated cell type (e.g., a protein or RNA which is up-regulated, down-regulated, turned on, or turned off in the cell type by the plant extract); administering compound(s) to the cell type, wherein the compound(s) are found in the plant extract; isolating protein or RNA from the compound(s) treated cell type; and identifying which of the compound(s) also result in the expression or suppression of the protein or RNA which is not present in the same concentration in the untreated cell type.

What is meant by “plant extract” is a collection of different natural compounds which are isolated from a plant (e.g., from the leaves, bark, fruits, flowers, seeds, or roots). Examples of such an extract is an extract from the tree ginkgo biloba. What is meant by “cell type” is either an isolated cell (e.g., derived from an organism such as a human or animal) or cells contained within a tissue or an organ from an organism. The cell type may be a normal cell or a diseased cell (e.g., a cell which is pathologically or physiologically different from its normal cell type, such as a tumor cell).

The plant extract and compound(s) may be administered to the cell type either in vitro or in vivo (e.g., to an intact animal from which the cell type is subsequently derived following treatment with the plant extract or compound(s)). When administered in vitro, the protein or RNA, for example, may be isolated immediately following or up to a day following administration of the plant extract or compound(s). When administered in vivo, the protein or RNA, for example, may be isolated immediately following or up to a day following the administration of the plant extract or compounds to the animal. The protein may remain within the cell type or secreted outside the cell type. Examples of such proteins include receptors, growth factors, enzymes, or transcription factors.

In another aspect, the invention features a method of determining the genomic response of a cell type to a plant extract, the method comprising the steps of: administering the plant extract to the cell type; isolating protein or RNA (e.g., messenger RNA) from the plant extract treated cell type; and comparing the protein or RNA isolated from the plant extract treated cell type to protein or RNA isolated from the untreated cell type.

In one embodiment, the method further comprises the step of identifying which of the protein or RNA isolated from the plant extract treated cell type is not present in the same concentration in the untreated cell type. In a further embodiment, the method further comprises sequencing the protein, RNA, or protein encoded by the RNA, identified from the plant extract treated cell type which is not present in the same concentration in the untreated cell type. In a still further embodiment, the method further comprises identifying the gene encoding the protein or RNA identified from the plant extract treated cell type which is not present in the same concentration in the untreated cell type.

In another embodiment, the cell type is associated with a known biological target (e.g., activation site) of the plant extract. For example, if the plant extract is known to treat asthma, cancer, circulation, or neurological disorders, then the cell type used is a lung cell, cancer cell, vascular cell, or neuronal cell, respectively.

Other features and advantages of the present invention will be apparent from the detailed description of the invention and from the claims.

### DETAILED DESCRIPTION OF THE INVENTION

It is believed that one skilled in the art can, based on the description herein, utilize the present invention to its fullest extent. The following specific embodiments are, therefore, to be construed as merely illustrative, and not limitative of the remainder of the disclosure in any way whatsoever.

Unless defined otherwise, all technical and scientific terms used herein have the same meaning as commonly understood by one of ordinary skill in the art to which this invention belongs. Also, all publications, patent applications, patents, and other references mentioned herein are incorporated by reference.

Plant Extracts and Its Individual Compounds

Plant extracts may be prepared by standard chemical extraction techniques (e.g., using water to extract hydrosoluble compounds, alcohols and acetone to extract liposoluble compounds, or mixtures thereof). The extract may then be further purified to decrease the number of compounds in the extract, or even isolate a single compound or compound class, by using standard techniques such as chromatography and crystallization. See, e.g., Ginkgolides—Chemistry, Biology, Pharmacology and Clinical Perspectives, edited by P. Braquet (J. R. Prous, Science Publishers, Barcelona, Spain 1988); Okabe, J. Chem. Soc. (c) p. 2201 (1967); and Nakauishi, Pure & Applied Chemistry 19:89 (1967).

Identification of Genomic Events by Isolating Protein

The target cell types, in culture, are harvested before and after exposure to a set concentration(s) of a plant extract (e.g., an extract of ginkgo biloba). The procedure may be similarly applied to cells from a target tissue or organ of an organism that has been exposed to a dose, or multiple doses, of a plant extract. The cells are lysed, and the proteins are solubilized in detergenized buffers (e.g., Tween 20, SDS, or NP-40 available from Sigma Chemicals, St. Louis, Mo.). The proteins are separated by two-dimensional gel electrophoresis, with isoelectric focusing in the first dimension and a gradient gel electrophoresis in the second dimension. See, e.g., Ausubel, A. M., et al., Current Protocols in Molecular Biology, pp 10.3.1-10.4.5 (John Wiley & Sons, 1987). This is an extremely powerful method for examining complex mixtures of proteins (e.g., as many as 1500 proteins may be resolved in a single 2-D Gel). See, e.g., O'Farrell, P. H., J. Biol. Chem. 250:4007-4021 (1975).

The pattern of protein expression may be visualized by Coomasie Blue staining (Ausubel, A. M., et al., Current Protocols in Molecular Biology, pp 10.6.1 (John Wiley & Sons, 1987)) or silver staining (Ausubel, A. M., et al., Current Protocols in Molecular Biology, pp 10.6.1-10.6.3 (John Wiley & Sons, 1987)). The cells may also be pulsed with a radio-labelled amino acids, e.g. S³⁵-Methionine (Amersham Corp., Arlington Heights, Ill.), and the pattern of labelled protein expression on the 2-D Gels is detected by autoradiography. In the case where the pattern of post-translationally modified proteins are examined, e.g., phosphorylated proteins, a radioactive phosphate donor, such as p³²-γATP (Amersham Corp., Arlington Heights, Ill.), is added to the incubation medium, and the pattern of phosphorylated protein on the 2-D gels may then be visualized by autoradiography. See, e.g., Hansen, K., et al., Electrophoresis 14:112-116 (1993); and Guy, R., Electrophoresis 15:417-440 (1994). In the latter case, unlabelled phosphoprotein expression may be visualized using phosphotyrosine or phosphoserine antibodies upon transfer of the gels to nitrocellulose. See, e.g., Ausubel, A. M., et al., Current Protocols in Molecular Biology, pp 10.7.1-10.8.6 (John Wiley & Sons, 1987).

Specific genomic events are detected by comparing the pattern of protein expression in cells before and after exposure to the plant extract. Specific protein spots on the 2-D gels that show either an enhancement or a reduction in expression, as evident from the intensity of the stain on the protein map, represents changes in genomic activity induced by components in the plant extract. The method is highly reproducible and can provide a means of collecting small amounts of extremely pure proteins for amino acid sequence analysis using standard techniques (i.e., Edmans amino-terminal degradation chemistry). See, e.g., Graven, et al., J. Biol. Chelm. 269:24446 (1994).

For proteins that are expressed at low quantities wherein sufficient amounts of the material may not be obtain for peptide sequencing, the protein spots may be excised from the 2-D gel and used directly for immunization in rabbits for antibody production. See Harlow, E. & Lane, D., Antibodies: A Laboratory Manual, Cold Spring Harbor Laboratory, 1988, pp. 61. The availability of an antibody allows the immunoaffinity purification of larger amounts of the regulated protein for peptide sequence identification.

From the peptide sequence, oligonucleotides may be synthesized based on redundant or most preferred genetic code for use as probes to screen a cDNA library to obtain the full length coding sequence of the gene(s). See, e.g., Sambrook, J., et al., Molecular Cloning: A Laboratory Manual, Book 2, (2nd Edition, Cold Spring Harbor Laboratory Press, 1989).

Identification of Genomic Events by Isolating mRNA

Another method for detecting changes in genomic events in response to a plant extract is to monitor messenger RNA (mRNA) changes in a cell (Liang, P. & Pardee, A. B., Science 257:967-971 (1992)). The cell will be exposed to a concentration of the plant extract in culture, or the cells from a target tissue or organ in an organism can be exposed locally or systemically with a dose or multiple doses of the plant extract for a defined period and subsequently extracted.

The mRNA will be prepared by routine total RNA extraction methods as described in Ausubel, A. M., et al., Current Protocols in Molecular Biology, pp 4.1.2-4.3.4 (John Wiley & Sons, 1987). From this preparation, Poly(A+) RNA may be prepared by oligo-dT chromatography (Aviv, H., & Leder, P., J. Mol. Biol. 134:743 (1972)) as described in Ausubel, A. M., et al., Current Protocols in Molecular Biology, pp 4.5.1-4.5.3 (John Wiley & Sons, 1987).

Two pools of mRNA are prepared, i.e., one from cells before and the other after exposure to the plant extract. Specific genomic events are represented in both pools depending on whether a suppression of gene activity or an activation of gene expression is induced by the plant extract. In the case where a gene is down-regulated in response to the plant extract, the mRNA will be present in higher quantities in the mRNA pool derived from the control cells. In the case where a gene is up-regulated in response to the plant extract, the mRNA will be present in higher quantities in the mRNA pool obtained from the plant extract treated cells. A number of techniques may be employed to identify mRNA populations that are up-regulated or down-regulated, e.g., subtractive hybridization or differential display.

(a) Subtraction Hybridization

Subtractive hybridization techniques (Lee, S. W., et al., Proc. Natl. Acad. Sci. 88:2825 (1991)) have been developed wherein the mRNA from one of the two pools is converted to first strand cDNA (antisense) by using oligo-dT primers, attached to either cellulose beads or free primers, and reverse transcriptase. See, e.g., Sambrook, J., et al., Molecular Cloning: A Laboratory Manual, Book 2, pp 10.46 (2nd Edition, Cold Spring Harbor Laboratory Press, 1989). In the case where the first strand is attached to cellulose beads, subtractive mRNA chromatography may be effected by passing the second mRNA pool through the column. mRNA represented in both pools will hybridize (i.e., mRNA from the second pool will hybridize to the antisense cDNA on the column and will be retained on the column). The flow through, which does not hybridize in the column, will contain mRNA species arising from an alteration in genomic activity from the effect of the plant extract on the cells. A cDNA bacteriophage library can then be prepared with mRNA from the flow-through fraction. See, e.g., Sambrook, J., et al., Molecular Cloning: A Laboratory Manual, Book 2, pp 8.11-8.45 (2nd Edition, Cold Spring Harbor Laboratory Press, 1989).

Specific clones not represented in the first mRNA pool are identified as non-hybridizing plaques if the library is screened with an P³²-end-labelled probes generated from the first pool. Other comparable strategies have also been described (Sambrook, J., et al., Molecular Cloning: A Laboratory Manual, Book 2, pp 10.41, 10.42 (2nd Edition, Cold Spring Harbor Laboratory Press, 1989).

The genes involved will be identified by DNA sequencing of the cDNA clones. See, e.g., Sambrook, J., et al., Molecular Cloning: A Laboratory Manual, Book 2, pp 13.3-13.20 (2nd Edition, Cold Spring Harbor Laboratory Press, 1989).

(b) mRNA Differential Display

This method is well described in the art. See, Liang, P., & Pardee, A. B., Science 257:967-970 (1992); Callard, D., et al., BioTechniques 16:1096-1103 (1994); Chen, Z., et al., BioTechniques 16:1003-1006 (1994); and Zhao, S., et al., BioTechniques 20:400-404 (1996). Partial cDNA sequences from mRNA prepared from the plant extract or treated and untreated cells are prepared by reverse transcription. See, e.g., Sambrook, J., et al., Molecular Cloning: A Laboratory Manual, Book 2, pp 10.46 (2nd Edition, Cold Spring Harbor Laboratory Press, 1989) and amplified by polymerase-chain reaction, e.g., using a 5′-T₁₁CA as a 3′-primer and a short (6-7 base) 5′-primer. The amplification reaction employs an α³⁵S-DATP label. The labelled, short PCR-amplified fragments from each mRNA pool are then fractionated (e.g., displayed) on a DNA sequencing gels. Bands not represented in equal intensity in one pool or the other are excised from the gel, reamplified, and used as a probe for screening a cDNA library, as described above, to identify the genes that are regulated by the plant extract.

Use

In this manner, a gene profile for a specific cell type in response to exposure to a particular plant extract of pharmacological interested may be catalogued without knowing the actual components in the plant extract. The utility of the plant extract can then be converted with the reported role of some of these genes in specific biological processes. In this manner, the therapeutic potential of the product may be extended beyond its known ethnopharmacological properties, or its known ethnopharmacological property can be genomically verified. Furthermore, similarly acting individual compound(s) in the extract can subsequently be identified as therapeutics based on the extract's activity on a certain set of genomic events.

### Other Embodiments

It is to be understood that while the invention has been described in conjunction with the detailed description thereof, that the foregoing description is intended to illustrate and not limit the scope of the invention, which is defined by the scope of the appended claims. Other aspects, advantages, and modifications are within the claims.

What is claimed is:

1. A method of identifying a pharmaceutical, said method comprising the steps of:
administering a plant extract to a cell type;
isolating protein or RNA from said plant extract treated cell type;
identifying which of said protein or RNA isolated from said plant extract treated cell type is not present in the same concentration in the untreated cell type;
administering compound(s) to said cell type, wherein said compound(s) are found in said plant extract;
isolating protein or RNA from said compound(s) treated cell type; and
identifying which of said compound(s) also result in the expression or suppression of said protein or RNA which is not present in the same concentration in said untreated cell type.

2. A method of claim 1, wherein said method comprises isolating protein from said plant extract treated cell type and said compound(s) treated cell type.

3. A method of claim 1, wherein said method comprises isolating RNA from said plant extract treated cell type and said compound(s) treated cell type.

4. A method of claim 3, wherein said RNA is messenger RNA.

5. A method of claim 1, wherein said plant extract and said compound(s) are administered in vitro.

6. A method of claim 1, wherein said plant extract and said compound(s) are administered in vivo.

7. A method of determining the genomic response of a cell type to a plant extract, said method comprising the steps of:
administering said plant extract to said cell type;
isolating protein or RNA from said plant extract treated cell type; and
comparing said protein or RNA isolated from said plant extract treated cell type to protein or RNA isolated from the untreated cell type.

8. A method of claim 7, wherein said method comprises isolating protein from said plant extract treated cell type.

9. A method of claim 7, wherein said method comprises isolating RNA from said plant extract treated cell type.

10. A method of claim 9, wherein said RNA is messenger RNA.

11. A method of claim 8, wherein said method further comprises the step of identifying which of said protein isolated from said plant extract treated cell type is not present in the same concentration in the untreated cell type.

12. A method of claim 11, wherein said method further comprises sequencing said protein identified from said plant extract treated cell type which is not present in the same concentration in said untreated cell type.

13. A method of claim 12, wherein said method further comprises identifying the gene encoding said protein identified from said plant extract treated cell type which is not present in the same concentration in said untreated cell type.

14. A method of claim 10, wherein said method further comprises the step of identifying which of said RNA isolated from said plant extract treated cell type is not present in the same concentration in the untreated cell type.

15. A method of claim 14, wherein said method further comprises sequencing said RNA identified from said plant extract treated cell type which is not present in the same concentration in said untreated cell type.

16. A method of claim 15, wherein said method further comprises identifying the gene identified by said RNA isolated from said plant extract treated cell type which is not present in the same concentration in said untreated cell type.

17. A method of claim 15, wherein said method further comprises sequencing the protein encoded by said RNA identified from said plant extract treated cell type which is not present in the same concentration in said untreated cell type.

18. A method of claim 7, wherein said plant extract is administered in vitro.

19. A method of claim 7, wherein said plant extract is administered in vivo.

20. A method of claim 7, wherein said cell type is associated with a known biological target of said plant extract.
